(19) United States
(12) Patent Application Publication
(10) Pub. No.: US 2025/0222210 A1
(43) Pub. Date: Jul. 10, 2025

(54) SPRAY DEVICE

(71) Applicant: TERUMO KABUSHIKI KAISHA, Tokyo (JP)

(72) Inventor: Kanako ANDO, Nakakoma-gun (JP)

(73) Assignee: TERUMO KABUSHIKI KAISHA, Tokyo (JP)

(21) Appl. No.: 19/092,001

(22) Filed: Mar. 27, 2025

Related U.S. Application Data
(63) Continuation of application No. PCT/JP2023/034181, filed on Sep. 21, 2023.

(30) Foreign Application Priority Data
Sep. 27, 2022 (JP) 2022-153185

(51) Int. Cl. A61M 11/00; A61M 15/08

(52) U.S. Cl. CPC A61M 11/007; A61M 15/08; A61M 2202/30; A61M 2205/073

(57) ABSTRACT

A spray device includes: a needled syringe including a barrel in which a drug solution storage chamber is formed, and a needle fixed to a distal end of the barrel; and a spray cap attachable to the distal end of the barrel. In a state in which the spray cap is attached to the distal end of the barrel, the spray cap accommodates the needle therein. The spray cap includes an atomization structure configured to change a drug solution supplied from the drug solution storage chamber through the needle into a mist form, and to spray the drug solution changed into the mist form.

### CROSS-REFERENCE TO RELATED APPLICATIONS

This is a bypass continuation of PCT Application No. PCT/JP2023/034181, filed on Sep. 21, 2023, which claims priority to Japanese Patent Application No. 2022-153185, filed on Sep. 27, 2022. The entire contents of these application are incorporated by reference herein.

### BACKGROUND

The present disclosure relates to a spray device that sprays a drug solution while changing the drug solution into a mist form.

The vaccine for nasal administration administered to the nasal cavity induces an IgA antibody different from that of a general vaccine for subcutaneous injection or vaccine for intramuscular injection. The IgA antibody has a cross-protection ability to prevent infection with a plurality of types of viruses. In addition, because the vaccine for nasal administration is not accompanied by pain at the time of administration, resistance of children is small. For the above reasons, there is a demand for more use of vaccines for nasal administration.

For example, the vaccine for subcutaneous injection is administered by subcutaneous injection from a needled syringe disclosed in JP 6479674 B2 and the like. On the other hand, the vaccine for nasal administration is changed into a mist form by the spray device, and is administered to the nasal cavity in this form. WO 2013/125555 A discloses a liquid atomization apparatus which is one type of spray device. The liquid atomization apparatus includes a plunger, similar to a needled syringe. The drug solution pressed by the plunger is atomized by the atomization structure. The atomized drug solution is sprayed from the spray port. The object to be sprayed is, for example, the nasal cavity.

### SUMMARY

As understood from JP 6479674 B2 and WO 2013/125555 A, the syringe (needled syringe) and the liquid atomization apparatus are separate, dedicated devices. That is, it is difficult to use a syringe as a liquid atomization apparatus, and it is also difficult to use a liquid atomization apparatus as a syringe. On the other hand, there is a demand for a general- purpose device that can be selectively used as either a syringe or a liquid atomization apparatus.

In a case where a spray device is to be manufactured from a needled syringe, it is conceivable to first remove the needle from the barrel and then attach the liquid atomization apparatus to the needle attachment portion of the barrel. However, such work is complicated.

An object of certain embodiments of the present disclosure is to solve the problem described above.

(1) One aspect of the present invention is a spray device including: a needled syringe including a barrel in which a drug solution storage chamber is formed, and a needle fixed to a distal end of the barrel; and a spray cap attachable to the distal end of the barrel, in which the spray cap accommodates the needle therein when the spray cap is attached to the distal end of the barrel, and the spray cap has an atomization structure that changes a drug solution supplied from the drug solution storage chamber through the needle into a mist form, and sprays the drug solution changed into the mist form.

According to this configuration, the needled syringe can be used as a syringe. In addition, in the case of configuring the spray device, the needled syringe is covered with the spray cap. The spray cap is capable of accommodating the needle therein. Therefore, when the spray cap is attached to the distal end of the barrel, it is not necessary to remove the needle from the barrel. In other words, the spray device can be configured by attaching the spray cap to the distal end of the barrel while the needle is attached to the barrel. Thus, according to the invention, a needled syringe can be selected as either a syringe or a spray device as needed by the user.

Moreover, when attaching the spray cap to the needled syringe, complicated work of detaching the needle from the barrel is unnecessary. That is, according to the present invention, the spray device is configured by a simple operation.

(2) In the spray device according to (1), the spray cap may include: an insertion port into which the distal end of the barrel is inserted; an inner chamber formed on a distal end side of the insertion port and into which the drug solution supplied from the drug solution storage chamber flows; and a partition that is interposed between the insertion port and the inner chamber and partitions the inner chamber into a space different from the insertion port. The distal end of the needle may pass through the partition and reach the inner chamber as the spray cap is attached to the distal end of the barrel, and the distal end of the needle may be disposed between a proximal end of the atomization structure and the partition in an axial direction of the spray cap.

According to this configuration, the drug solution is introduced into the inner chamber or the atomization structure of the spray cap, and then sprayed from the spray port. That is, the drug solution path in the spray cap is only the inner chamber and the spray port. As described above, in the present aspect, the drug solution path in the spray cap can be shortened. Therefore, the amount of the drug solution in the drug solution path is small. Therefore, the amount of the drug solution remaining in the drug solution path (inside the spray cap) after spraying the drug solution decreases.

(3) In the spray device according to (2), the partition may be an elastic body.

The elastic body is relatively flexible. Therefore, in this case, the distal end of the needle easily penetrates the partition and reaches the inner chamber. That is, according to this configuration, it is easy to cause the distal end of the needle to reach the inner chamber.

(4) In the spray device according to (3), the partition may include an insertion portion into which the needle is inserted and removed as the spray cap is attached to and detached from the barrel.

In this case, because the needle passes through the insertion portion, the needle does not penetrate the partition. Therefore, there is no concern that the lumen of the needle is blocked by the fragment of the partition.

(5) In the spray device according to (4), the insertion portion may open as the needle is inserted into the inner chamber and close as the needle is detached from the inner chamber.

In this case, the insertion portion opened as the needle is inserted into the inner chamber tries to return to the position before the needle is inserted. Therefore, the inner wall of the insertion portion comes into close contact with the peripheral wall of the needle passed through the insertion portion. Therefore, leakage of the drug solution flowing into the inner chamber to the needle accommodating part is avoided.

(6) In the spray device according to any one of (2) to (5), the partition may be a disk-shaped body.

In this case, the spray cap is lightweight. In addition, in a case where the partition is a disk-shaped body, it is relatively easy for the needle to penetrate the partition.

(7) In the spray device according to any one of (2) to (6), the partition may be a filling portion that fills a space between the inner chamber and the insertion port.

In this case, the partition reliably blocks the drug solution. Therefore, backflow of the drug solution from the inner chamber to the insertion port is avoided. Therefore, leakage of the drug solution from the insertion port is avoided.

(8) In the spray device according to any one of (2) to (7), the partition may have a tapered hole whose diameter expands in a tapered shape from the inner chamber toward the insertion port.

The tapered hole guides the distal end of the needle to a predetermined position of the partition. For example, an insertion hole is formed at a predetermined position. In this case, it is easy to align the position of the needle with the position of the insertion hole. Therefore, it is also easy to pass the needle through the insertion hole.

(9) In the spray device according to (1), the spray cap may include: an insertion port into which the distal end of the barrel is inserted; and an inner chamber which is formed on a distal end side of the insertion port and into which the drug solution supplied from the drug solution storage chamber flows, and the spray cap may be a variable capacity container capable of changing a capacity of the inner chamber.

By making the capacity of the inner chamber variable, the drug solution can be easily sprayed regardless of whether the drug solution has a high viscosity or a low viscosity.

(10) In the spray device according to (9), the variable capacity container may include: a partition that is interposed between the insertion port and the inner chamber and partitions the inner chamber into a space different from the insertion port; a first segment provided with the atomization structure; and a second segment provided with the partition, the spray cap may be configured by connecting the first segment to the second segment in a relatively movable manner, and the first segment may move relative to the second segment to change a capacity of the inner chamber.

According to this configuration, it is easy to change the capacity of the inner chamber to a desired capacity.

(11) In the spray device according to any one of (1) to (10), the spray cap may include: an insertion port into which the distal end of the barrel is inserted; an inner chamber which is formed on a distal end side of the insertion port and into which the drug solution supplied from the drug solution storage chamber flows; and a core provided with the atomization structure, and the core may be accommodated in the inner chamber.

An example of such a core is a porous body such as a sponge. Another example of the core is a member in which a spiral groove is formed. Still another example of the core is a member in which an orifice is formed. The drug solution changes from liquid to mist in the process of flowing along the core. According to this configuration, it is easy to change the drug solution into a mist form.

(12) In the spray device according to (11), the core may be positioned and fixed to the spray cap within the inner chamber.

In this case, it is easy to change the drug solution having a predetermined viscosity into a mist form.

(13) In the spray device according to any one of (1) to (10), the atomization structure may be formed in a spray port for spraying the atomized drug solution in the spray cap.

In this configuration, it is not particularly necessary to accommodate the core in the inner chamber. Therefore, it is easy to manufacture the spray cap.

### BRIEF DESCRIPTION OF THE DRAWINGS

FIG. 1 is a schematic side sectional view of a spray device according to a first embodiment of the present invention as viewed along a longitudinal direction.

FIG. 2 is a schematic side sectional view of a needled syringe and a protector as viewed along the longitudinal direction.

FIG. 3 is a schematic side sectional view of a spray cap constituting the spray device.

FIG. 4 is a schematic side sectional view of a spray cap having an atomization structure different from the atomization structure illustrated in FIGS. 1 and 3.

FIG. 5 is a schematic perspective view illustrating a state in which the distal end of the needle penetrates a partition.

FIG. 6A is a schematic perspective view of a partition in which an insertion portion is formed. FIG. 6B is a schematic perspective view illustrating a state in which the needle is inserted through the insertion portion of the partition in FIG. 6A.

FIG. 7 is a schematic side sectional view of a spray device according to a second embodiment of the present invention as viewed along the longitudinal direction.

FIG. 8 is a schematic side sectional view of a spray cap in a different shape from the spray cap illustrated in FIG. 7.

FIG. 9A is a schematic side sectional view of the spray cap when the volume of the inner chamber is large. FIG. 9B is a schematic side sectional view of the spray cap when the volume of the inner chamber is small.

### DETAILED DESCRIPTION

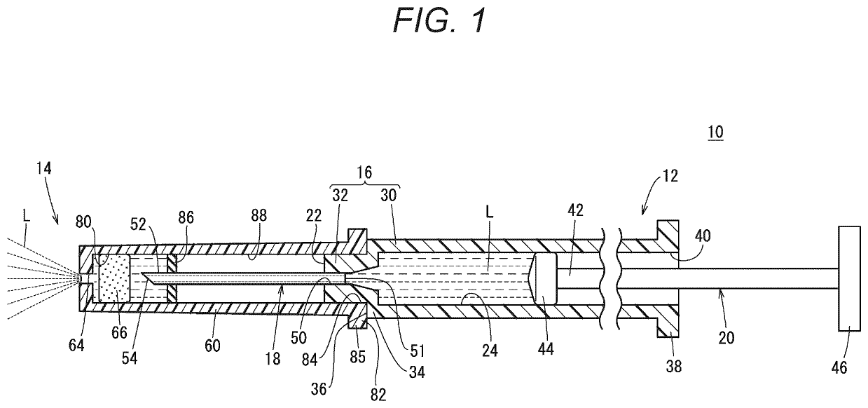

FIG. 1 is a schematic side sectional view of a spray device 10 according to a first embodiment as viewed along a longitudinal direction. The spray device 10 includes a needled syringe 12 and a spray cap 14.

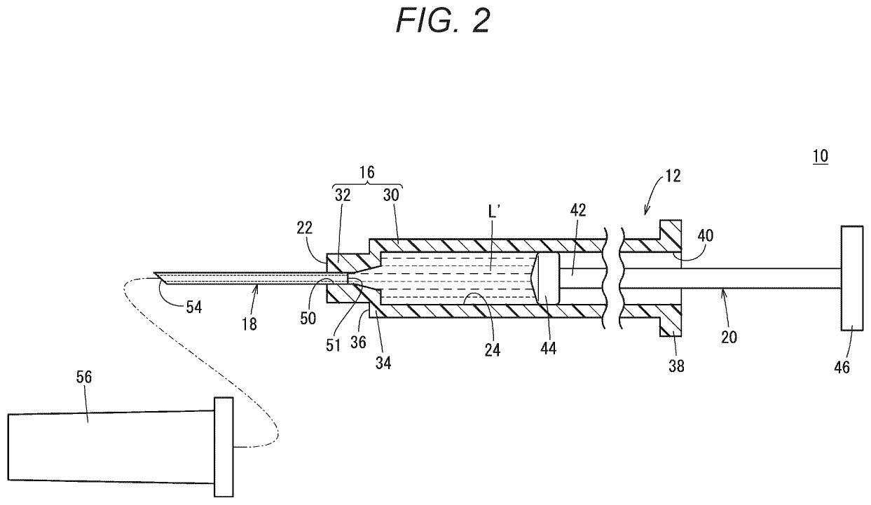

The needled syringe 12 will be schematically described with reference to FIGS. 1 and 2. The needled syringe 12 has a barrel 16, a needle 18, and a plunger 20. The barrel 16 is a transparent or translucent cylindrical body. In the barrel 16, a cap-side end portion 22 is on the spray cap 14 side. Hereinafter, the spray cap 14 side of the barrel 16 may be referred to as a “distal end side” or a “distal end direction”. In addition, the side of the barrel 16 opposite to the spray cap 14 may be referred to as a “proximal end side” or a “proximal end direction”. A drug solution storage chamber 24 is formed inside the barrel 16.

The barrel 16 has a main body portion 30 and a cylindrical protrusion 32. The main body portion 30 is formed of a relatively long cylindrical body extending along the axial direction. The cylindrical protrusion 32 further protrudes toward the distal end side from the cap-side end portion 22 which is the distal end of the main body portion 30. The cylindrical protrusion 32 is formed of a short cylindrical body. The outer diameter of the cylindrical protrusion 32 is smaller than the outer diameter of the main body portion 30. Based on this outer diameter difference, a stepped portion 36 is formed between the main body portion 30 and the cylindrical protrusion 32.

An opening 40 is formed in a plunger side end portion 38 which is a proximal end of the main body portion 30. A barrel-side end portion 42 which is a distal end of the plunger 20 is inserted into the drug solution storage chamber 24 from the opening 40. A gasket 44 is attached to the barrel-side end portion 42 of the plunger 20. An operation end portion 46, which is the proximal end of the plunger 20, protrudes from the opening 40 of the main body portion 30 to the proximal end side.

A drug solution L (see FIG. 1) or a drug solution L′ (see FIG. 2) is stored in the drug solution storage chamber 24 of the barrel 16. Examples of the drug solution L include a vaccine for nasal administration, and examples of the drug solution L′ include a vaccine for subcutaneous injection or a vaccine for intramuscular injection. As the user pushes the operation end portion 46 of the plunger 20 toward the distal end side, the plunger 20 moves toward the distal end side. At this time, the drug solution L or the drug solution L′ in the drug solution storage chamber 24 is pressed by the gasket 44. In the spray device 10, as will be described later, the drug solution L is sprayed from the spray cap 14 along with this pressing.

A needle hole 50 extending along the axial direction of the barrel 16 is formed in the cylindrical protrusion 32. The needle hole 50 communicates with the drug solution storage chamber 24. A barrel-side end portion 51 which is the proximal end of the needle 18 is inserted into the needle hole 50. A cap-side end portion 52 which is the distal end of the needle 18 is exposed from the needle hole 50. A lumen 54 is formed in the needle 18. As the plunger 20 moves toward the spray cap 14, the drug solution L or the drug solution L′ in the needle hole 50 is pushed into the lumen 54 of the needle 18. The drug solution L or the drug solution L′ further flows through the lumen 54.

Before the spray cap 14 is attached to the needled syringe 12, a protector 56 illustrated in FIG. 2 is attached to the cap-side end portion 22 of the needled syringe 12. The protector 56 covers the needle 18 and the cylindrical protrusion 32. Thus, the needle 18 is protected by the protector 56.

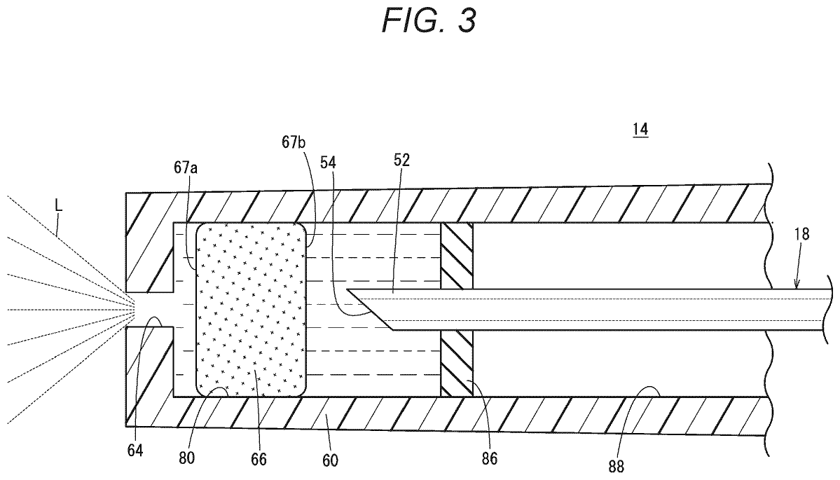

The spray device 10 is assembled by attaching the spray cap 14 (see FIG. 1) to the needled syringe 12 instead of the protector 56. Next, the spray cap 14 will be described with reference to FIGS. 1 and 3.

FIG. 3 is a schematic side sectional view of the spray cap 14. The spray cap 14 has a cap body 60. A spray port 64 is formed at the distal end of the cap body 60. In this aspect, a core 66 is provided inside the cap body 60. The core 66 has an atomization structure that changes the drug solution L into a mist form. In this manner, the cap body 60 is provided with the atomization structure. A distal end 67a of the core 66 faces the spray port 64 side. A proximal end 67b of the core 66 faces the barrel 16 side. The outer peripheral wall of the core 66 is in contact with the inner peripheral wall inside the cap body 60. The core 66 is thereby positioned and fixed inside the cap body 60.

Alternatively, the core 66 may be inserted into the cap body 60 without positioning and fixing the core 66 with respect to the cap body 60. In this case, the core 66 is not restrained by the cap body 60. Therefore, the core 66 is movable relative to the cap body 60. Therefore, in a case where a needle having a length different from that of the needle 18 is used, the position of the core 66 can be changed according to the length of the needle.

An example of the core 66 is a porous body such as a sponge. Another example of the core 66 is a member in which a spiral groove is formed. Still another example of the core 66 is a member in which an orifice is formed. In any core 66, the drug solution L changes to a mist form in the course of the drug solution L passing through the core 66.

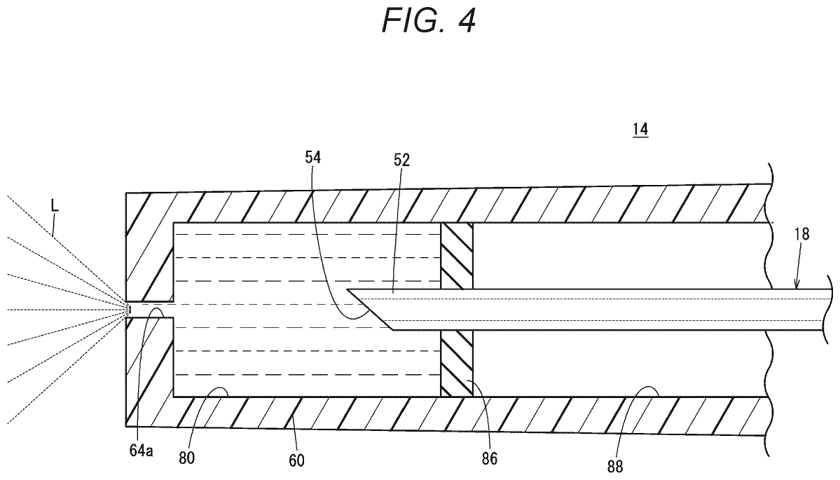

As illustrated in FIG. 4, a spray port 64a may have an atomization structure. In this case, the spray port 64a is formed as an orifice.

Returning to FIG. 3, a partition 86 is provided inside the cap body 60. The partition 86 is closer to the proximal end side than the core 66. In this case, the partition 86 is a disk-shaped body. The side peripheral wall of the partition 86 is joined to the inner peripheral wall of the cap body 60. The partition 86 is made of, for example, an elastic body.

The partition 86 divides the interior of the spray cap 14 into an inner chamber 80 and a needle accommodating part 88. In the needle accommodating part 88, a proximal end facing the barrel 16 side is an insertion port 84 illustrated in FIG. 1. In other words, in the cap body 60, the insertion port 84 is formed in a proximal end 82 facing toward the barrel 16 side. The insertion port 84 is an opening into which the cylindrical protrusion 32 is inserted.

The proximal end 82 is provided with a flange portion 85 protruding radially outward. A proximal end surface of the flange portion 85 facing the barrel 16 side abuts on the stepped portion 36 of the barrel 16. With this contact, the insertion of the cylindrical protrusion 32 into the insertion port 84 is completed, and the spray cap 14 is attached to the barrel 16. The inner diameter of the insertion port 84 is slightly smaller than or substantially the same as the outer diameter of the cylindrical protrusion 32. Therefore, the inner peripheral wall of the insertion port 84 (the proximal end of the spray cap 14) pushes the cylindrical protrusion 32 inward in the diametrical direction. This prevents the spray cap 14 attached to the barrel 16 from falling off the cylindrical protrusion 32.

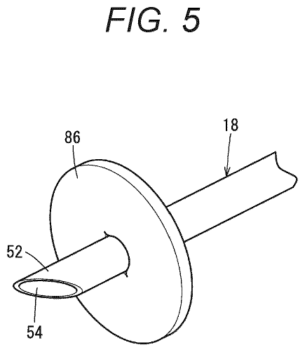

When the spray cap 14 is attached to the cylindrical protrusion 32, the cap-side end portion 52 of the needle 18 penetrates the partition 86 as illustrated in FIG. 5. In other words, the cap-side end portion 52 of the needle 18 penetrates the partition 86 and reaches the inner chamber 80. Accordingly, the drug solution storage chamber 24 of the barrel 16 and the inner chamber 80 of the spray cap 14 communicate with each other via the needle hole 50 and the lumen 54 of the needle 18.

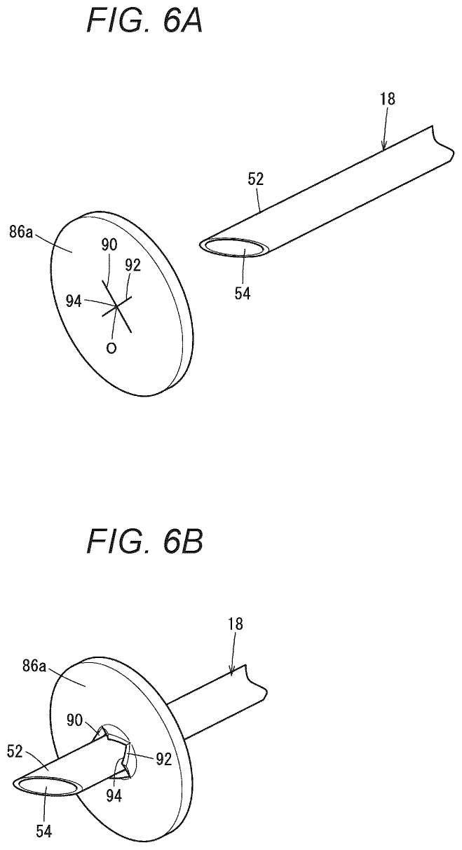

FIG. 6A is a schematic perspective view of a partition 86a according to a modification. Two slits 90 and 92 are formed in advance in the partition 86a. The two slits 90 and 92 intersect at the center O of the partition 86a so as to be substantially orthogonal to each other. Thus, an insertion portion 94 is formed. In the vicinity of the insertion portion 94, the partition 86a is easily bent. Therefore, when the spray cap 14 is attached to the cylindrical protrusion 32, the vicinity of the insertion portion 94 is pressed by the needle 18 and bent toward the distal end side as illustrated in FIG. 6B. That is, the insertion portion 94 opens as the needle 18 is inserted into the inner chamber 80.

The open insertion portion 94 tries to return to the closed position by elastic action. Therefore, the insertion portion 94 is in close contact with the peripheral wall of the needle 18. Therefore, the periphery of the needle 18 is sealed.

Conversely, when the spray cap 14 is detached from the cylindrical protrusion 32, the vicinity of the insertion portion 94 returns to the original shape illustrated in FIG. 6A by the elastic action. That is, the insertion portion 94 is closed as the needle 18 is detached from the inner chamber 80. In this manner, the partition 86a has the insertion portion 94 into which the needle 18 is inserted and removed as the spray cap 14 is attached to and detached from the barrel 16. In this case, the insertion portion 94 opens as the needle 18 is inserted into the inner chamber 80, and closes as the needle 18 is detached from the inner chamber 80.

The spray device 10 according to the first embodiment is basically configured as described above. Next, operational effects of the spray device 10 will be described.

As illustrated in FIG. 2, the needled syringe 12 is stored in a state where the protector 56 is attached to the cap-side end portion 22 of the needled syringe 12. In a case where the needled syringe 12 is used as a syringe, the user first removes the protector 56 from the needled syringe 12. Next, the user inserts the needle 18 into a vial (not illustrated), and in this state, pulls the operation end portion 46 of the plunger 20 in the proximal end direction from the drug solution storage chamber 24. As a result, the drug solution L′ in the vial moves to the drug solution storage chamber 24 of the barrel 16.

Next, the user pierces the skin of the patient with the needle 18, and in this state, pushes the operation end portion 46 of the plunger 20 in the distal end direction of the main body portion 30. As a result, the drug solution L′ in the drug solution storage chamber 24 is injected into the body of the patient.

In a case where the needled syringe 12 is used as the spray device 10, the user first removes the protector 56 from the needled syringe 12. Next, the user inserts the needle 18 into the vial, and in this state, pulls the plunger 20 in the proximal end direction from the drug solution storage chamber 24. As a result, the drug solution L in the vial moves to the drug solution storage chamber 24 of the barrel 16. The drug solution L in this case is typically a vaccine for nasal administration.

The user then attaches the spray cap 14 to the barrel 16 with the needle 18 attached, as illustrated in FIG. 3. Specifically, the cylindrical protrusion 32 is inserted into the insertion port 84 of the spray cap 14. As a result, the cap-side end portion 52 of the needle 18 is inserted into the needle accommodating part 88 and abuts on the partition 86. When the needle 18 further moves relatively toward the distal end side, the sharp cap-side end portion 52 of the needle 18 penetrates the partition 86 (see FIG. 5). Because the partition 86 is made of a flexible elastic body, the cap-side end portion 52 of the needle 18 easily penetrates the partition 86. The periphery of the needle 18 is sealed by the partition 86.

As a result, the cap-side end portion 52 of the needle 18 reaches the inner chamber 80. Therefore, the drug solution storage chamber 24 and the inner chamber 80 communicate with each other via the needle hole 50 and the lumen 54 of the needle 18. As the proximal end surface of the flange portion 85 comes into contact with the stepped portion 36 of the barrel 16, the insertion of the cylindrical protrusion 32 into the insertion port 84 is terminated. In this state, the cap-side end portion 52 of the needle 18 is located in the inner chamber 80 and the majority of the needle 18 is located in the needle accommodating part 88 of the spray cap 14. When the spray device 10 is viewed from the spray port 64, the atomization structure (core 66), the cap-side end portion 52 of the needle 18, and the partition 86 are arranged in this order from the distal end side toward the proximal end side. In other words, the cap-side end portion 52 which is the distal end of the needle 18 is positioned between the distal end 67a of the core 66 and the distal end surface of the partition 86.

The cap-side end portion 52 of the needle 18 may be inserted into the core 66. However, the cap-side end portion 52 of the needle 18 stops at a position not penetrating the core 66. That is, also in this case, the cap-side end portion 52 which is the distal end of the needle 18 is positioned between the distal end 67a of the core 66 and the distal end surface of the partition 86.

In the partition 86a illustrated in FIG. 6A, the cap-side end portion 52 of the needle 18 pushes the vicinity of the intersection of the two slits 90 and 92. Because the partition 86a is made of a flexible elastic body, the vicinity of the intersection of the two slits 90 and 92 is easily bent in the distal end direction as illustrated in FIG. 6B. That is, also in this aspect, the cap-side end portion 52 of the needle 18 easily reaches the inner chamber 80.

When exemplifying a case where the drug solution L is a vaccine for nasal administration, the user inserts the distal end formed with the spray port 64 into the nasal cavity of the patient in the spray cap 14. Next, the user pushes the plunger 20 toward the distal end side of the main body portion 30. Accordingly, the drug solution L in the drug solution storage chamber 24 flows into the lumen 54 of the needle 18 through the needle hole 50. When the plunger 20 is further pushed toward the distal end side by the user, the drug solution L in the lumen 54 flows into the inner chamber 80 of the spray cap 14, and is temporarily stored in the space between the core 66 and the partition 86 in the inner chamber 80.

When the plunger 20 is further pushed toward the distal end side by the user, the drug solution L in the space passes through the core 66. In this aspect, because the core 66 has an atomization structure, the drug solution L changes into a mist form in the process of passing through the core 66. The atomized drug solution L is sprayed from the spray port 64 to the nasal cavity. The drug solution L adheres to the mucous membrane in the nasal cavity and is taken into the body of the patient via the mucous membrane.

As described above, according to the first embodiment, the spray device 10 can be configured by replacing the protector 56 with the spray cap 14 while the needle 18 is attached to the barrel 16. That is, it is not necessary to remove the needle 18 from the needled syringe 12 when configuring the spray device 10. Therefore, it is easy to obtain the spray device 10 from the needled syringe 12.

Moreover, as will be appreciated from the above, the needled syringe 12 may be selectively used as either the syringe or the spray device 10 as needed by the user. That is, versatility of the needled syringe 12 is improved.

Here, in the spray device according to the prior art, a drug solution path from the barrel to the spray port is a relatively long distance in the spray cap. Therefore, the amount of the drug solution in the drug solution path is relatively large. In this case, the absolute amount of the drug solution remaining in the drug solution path is relatively large after the user pushes the plunger to spray the drug solution. Therefore, a relatively large amount of drug solution needs to be discarded.

On the other hand, the drug solution path in the spray cap 14 in the first embodiment is only the inner chamber 80 and the spray port 64. Therefore, the drug solution path in the spray cap 14 is a relatively short distance. Therefore, the absolute amount of the drug solution L remaining in the drug solution path decreases. That is, according to the first embodiment, it is possible to spray substantially the entire amount of the drug solution L transferred from the vial to the drug solution storage chamber 24 of the barrel 16. As a result, the amount of the drug solution L to be discarded can be reduced.

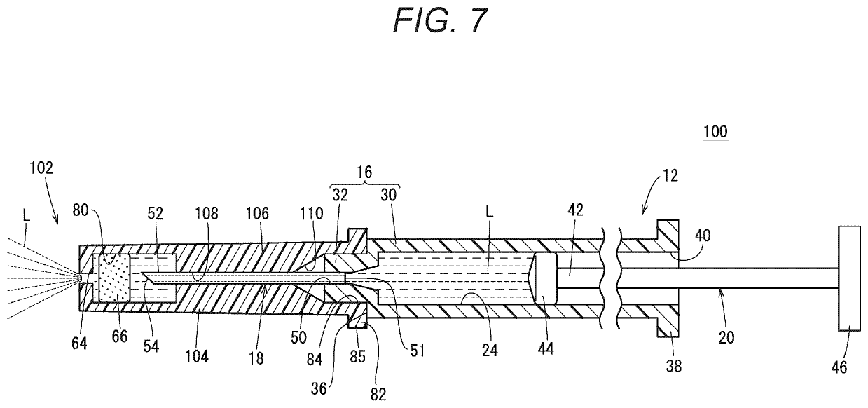

Next, a spray device 100 according to a second embodiment will be described with reference to FIG. 7. Note that components of the needled syringe 12 in the second embodiment are denoted by the same reference numerals as the components of the needled syringe 12 in the first embodiment, and a detailed description thereof will be omitted. Further, in a case where the components of a spray cap 102 are the same as the components of the spray cap 14, the same reference numerals are basically given.

The spray device 100 includes a needled syringe 12 and a spray cap 102. The spray cap 102 has a cap body 104. A partition 106 is provided inside the cap body 104. The partition 106 is a filling portion for filling a space between the inner chamber 80 and the insertion port 84. An insertion hole 108 through which the needle 18 is inserted is formed in advance in the partition 106. The insertion hole 108 is continuous with the inner chamber 80. The insertion hole 108 serves as a needle accommodating part that accommodates the needle 18. The diameter of the insertion hole 108 is substantially equal to the outer diameter of the needle 18.

A tapered hole 110 is connected to the insertion hole 108. The diameter of the tapered hole 110 increases in a tapered shape from the inner chamber 80 toward the insertion port 84. The tapered hole 110 guides the needle 18 to the insertion hole 108.

A core 66 is provided inside the cap body 104. The core 66 has an atomization structure as in the first embodiment.

Next, operational effects of the spray device 100 according to the second embodiment will be described.

In a case where the needled syringe 12 is used as the spray device 100, the user removes the protector 56 from the needled syringe 12 (see FIG. 2), and then transfers the drug solution L (vaccine for nasal administration, etc.) in the vial to the drug solution storage chamber 24 of the barrel 16, as in the first embodiment. Next, as illustrated in FIG. 7, the user attaches the spray cap 102 to the barrel 16 to which the needle 18 is attached. Specifically, the cylindrical protrusion 32 is inserted into the insertion port 84 of the spray cap 102. In this case, when the cap-side end portion 52 of the needle 18 abuts against the wall of the tapered hole 110, the cap-side end portion 52 of the needle 18 is guided by the wall of the tapered hole 110 toward the insertion hole 108. When the cap-side end portion 52 of the needle 18 and the insertion hole 108 are aligned, the cap-side end portion 52 of the needle 18 enters the insertion hole 108.

The cap-side end portion 52 of the needle 18 passes through the insertion hole 108 and reaches the inner chamber 80. Therefore, the drug solution storage chamber 24 and the inner chamber 80 communicate with each other via the needle hole 50 and the lumen 54 of the needle 18. As the end surface of the flange portion 85 on the barrel 16 side comes into contact with the stepped portion 36 of the barrel 16, the insertion of the cylindrical protrusion 32 into the insertion port 84 is terminated. In this state, the cap-side end portion 52 of the needle 18 is located in the inner chamber 80 and the majority of the needle 18 is located in the insertion hole 108 of the spray cap 14. When the spray device 100 is viewed from the spray port 64, the atomization structure (core 66), the cap-side end portion 52 of the needle 18, and the partition 106 (filling portion) are arranged in this order from the distal end side toward the proximal end side. The cap-side end portion 52 of the needle 18 may be inserted into the core 66. However, the cap-side end portion 52 of the needle 18 stops at a position not penetrating the core 66.

In a case where the drug solution L is a vaccine for nasal administration, the user inserts the distal end formed with the spray port 64 into the nasal cavity of the patient in the spray cap 102. Next, the user pushes the plunger 20 toward the distal end side of the barrel 16. Accordingly, the drug solution L in the drug solution storage chamber 24 flows into the lumen 54 of the needle 18 through the needle hole 50. When the plunger 20 is further pushed toward the distal end side by the user, the drug solution L in the lumen 54 flows into the inner chamber 80 of the spray cap 14, and is temporarily stored in the space between the core 66 and the partition 106 in the inner chamber 80.

When the plunger 20 is further pushed toward the distal end side by the user, the drug solution L in the space passes through the core 66. In this aspect, because the core 66 has an atomization structure, the drug solution L changes into a mist form in the process of passing through the core 66. The atomized drug solution L is sprayed from the spray port 64 to the nasal cavity. The drug solution L adheres to the mucous membrane in the nasal cavity and is taken into the body of the patient via the mucous membrane.

As described above, also in the second embodiment, the spray device 100 can be configured by replacing the protector 56 with the spray cap 102 while the needle 18 is attached to the barrel 16. That is, it is not necessary to remove the needle 18 from the needled syringe 12 in order to configure the spray device 100. Therefore, it is easy to obtain the spray device 100 from the needled syringe 12.

Also in the second embodiment, the drug solution path in the spray cap 102 is only the inner chamber 80 and the spray port 64. Therefore, the drug solution path in the spray cap 102 is a relatively short distance. Therefore, the absolute amount of the drug solution L remaining in the drug solution path decreases. In this way, also in the second embodiment, it is possible to spray substantially the entire amount of the drug solution L transferred from the vial to the drug solution storage chamber 24 of the barrel 16. As a result, the amount of the drug solution L to be discarded can be reduced.

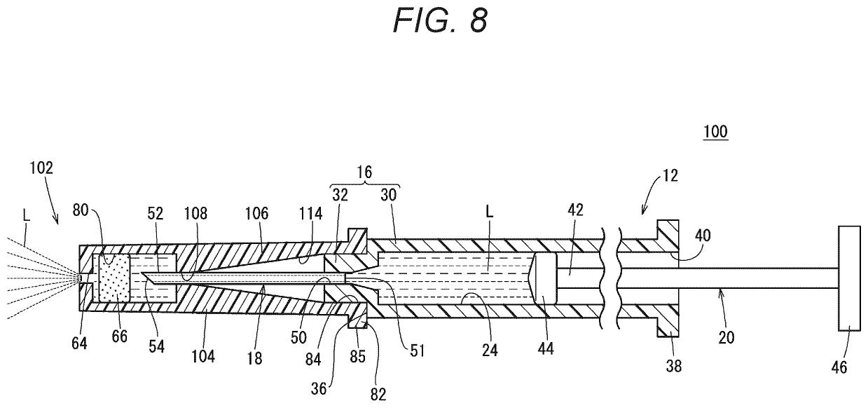

As illustrated in FIG. 8, instead of the tapered hole 110, a tapered hole 114 longer than the insertion hole 108 may be formed.

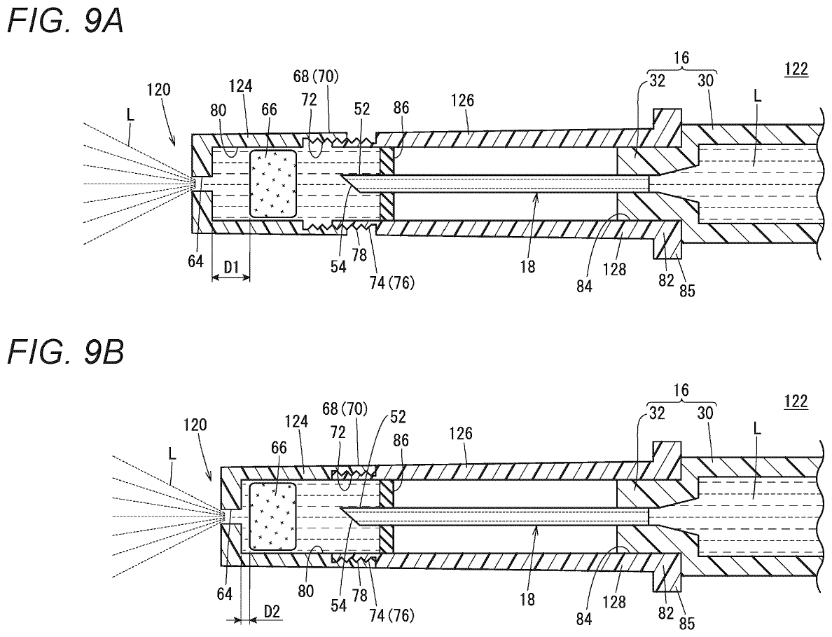

Incidentally, the viscosity of the drug solution L varies depending on the type of the drug solution L. In order to make it possible to spray the drug solution L regardless of the level of the viscosity of the drug solution L, a spray cap 120 is preferably a variable capacity container as illustrated in FIGS. 9A and 9B. A spray device 122 according to this aspect will be described. In this aspect, a case where the core 66 inserted into the spray cap 120 is movable relative to the spray cap 120 will be exemplified.

In this case, the spray cap 120 has a first segment 124 and a second segment 126. In the first segment 124, the outer diameter of a proximal end 68 located on the proximal end side is equal to the outer diameters of the other portions. On the other hand, the inner diameter of the proximal end 68 of the first segment 124 is larger than the inner diameters of the other portions. Based on this inner diameter difference, a first thin portion 70 is formed at the proximal end 68 of the first segment 124. A first screw portion 72 is provided on an inner peripheral wall of the first thin portion 70. The core 66 is closer to the distal end side than the first thin portion 70.

In the second segment 126, the inner diameter of a distal end 74 facing the first segment 124 is equal to the inner diameters of the other portions. On the other hand, the outer diameter of the distal end 74 of the second segment 126 is smaller than the outer diameters of the other portions. A second thin portion 76 is formed at the distal end 74 of the second segment 126 based on the outer diameter difference. A second screw portion 78 is provided on an outer peripheral wall of the second thin portion 76. The second screw portion 78 is screwed into the first screw portion 72. By this screwing, the spray cap 120 is configured by connecting the first segment 124 and the second segment 126.

The screwing position of the first segment 124 with respect to the second segment 126 can be appropriately changed. As illustrated in FIGS. 9A and 9B, the insertion depth of the second thin portion 76 into the first thin portion 70 is also changed in accordance with the change of the screwing position. As illustrated in FIG. 9A, when the insertion depth of the second thin portion 76 into the first thin portion 70 is small, the volume (capacity) of the inner chamber 80 described later increases. Therefore, the drug solution storage amount in the inner chamber 80 increases. On the other hand, as illustrated in FIG. 9B, when the insertion depth of the second thin portion 76 into the first thin portion 70 is large, the volume (capacity) of the inner chamber 80 decreases. Therefore, the drug solution storage amount in the inner chamber 80 decreases. As described above, the spray cap 120 is a variable capacity container in which the capacity of the inner chamber 80 can be changed. The insertion port 84 is formed in a proximal end 128 facing the barrel 16 side in the second segment 126.

In this configuration, in a case where the viscosity of the drug solution L is low, as illustrated in FIG. 9A, the insertion depth of the second thin portion 76 into the first thin portion 70 decreases. In this case, a relatively large amount of the drug solution L can be stored in the inner chamber 80.

Because the drug solution L having a low viscosity has a small flow resistance, the drug solution L relatively easily passes through the core 66. Therefore, the pressing force that the core 66 receives from the drug solution L is relatively small. Therefore, because the movement amount of the core 66 in the inner chamber 80 is small, the spray port 64 and the distal end surface of the core 66 are relatively separated from each other. This makes it easy to spray the low-viscosity drug solution L from the spray port 64. A separation distance between the spray port 64 and the distal end surface of the core 66 is D1.

On the other hand, in a case where the viscosity of the drug solution L is high, as illustrated in FIG. 9B, the insertion depth of the second thin portion 76 into the first thin portion 70 is increased. In this case, the drug solution storage amount in the inner chamber 80 is relatively small.

The drug solution L having a high viscosity has high flow resistance. Therefore, the core 66 receives a relatively large pressing force from the drug solution L. Therefore, the movement amount of the core 66 in the inner chamber 80 increases, and the spray port 64 and the distal end surface of the core 66 are relatively close to each other. This makes it easy to spray the drug solution L having high viscosity from the spray port 64. The separation distance between the spray port 64 and the distal end surface of the core 66 is D2. D2 is smaller than D1.

As described above, the spray cap 120 is a variable capacity container in which the capacity of the inner chamber 80 can be changed, so that the atomized drug solution L can be easily sprayed regardless of the level of the viscosity of the drug solution L. The connection between the first segment 124 and the second segment 126 may be a slide lock type.

In the first embodiment and the second embodiment described above, a case where the spray devices 10 and 100 are used as devices for nasal administration is exemplified. However, the application of the spray devices 10 and 100 is not particularly limited to devices for nasal administration. Other applications for spray devices include laryngeal atomizers.

The operational effects of the first embodiment and the second embodiment described above are summarized as follows.

The spray device 10 includes a needled syringe 12 having a barrel 16 in which a drug solution storage chamber 24 is formed and a needle 18 fixed to a cap-side end portion 22 (distal end) of the barrel 16, and a spray cap 14 attachable to the distal end of the barrel 16. The spray cap 14 accommodates the needle 18 therein when the spray cap 14 is attached to the distal end of the barrel 16. The spray cap 14 has an atomization structure that changes the drug solution L supplied from the drug solution storage chamber 24 via the needle 18 into a mist form. The spray device 10 sprays the drug solution L that has changed to a mist form.

As will be appreciated from the above, the spray cap 14 of the spray device 10 is capable of accommodating the needle 18 therein. Therefore, when the spray cap 14 is attached to the distal end of the barrel 16, it is not necessary to remove the needle 18 from the barrel 16. In other words, the spray cap 14 can be attached to the distal end of the barrel 16 while the needle 18 is attached to the barrel 16. The spray device 10 is configured by this attachment.

The needled syringe 12 before attachment of the spray cap 14 can be used as a syringe. That is, in this case, the needled syringe 12 can be selectively used as either the syringe or the spray device 10 as needed by the user. Therefore, versatility of the needled syringe 12 is improved.

Moreover, when the needled syringe 12 is used as the spray device 10, the operation of detaching the needle 18 from the barrel 16 is unnecessary. That is, according to the present invention, the spray device 10 can be obtained by a simple operation.

The spray cap 14 includes an insertion port 84 into which the distal end of the barrel 16 is inserted, an inner chamber 80 that is formed on the distal end side of the insertion port 84 and into which the drug solution L supplied from the drug solution storage chamber 24 flows, and a partition 86 that is interposed between the insertion port 84 and the inner chamber 80 and partitions the inner chamber 80 from the insertion port 84. As the spray cap 14 is attached to the distal end of the barrel 16, the distal end (cap-side end portion 52) of the needle 18 passes through the partition 86 and reaches the inner chamber 80. In the axial direction of the spray cap 14, the cap-side end portion 52 of the needle 18 is disposed between the proximal end of the atomization structure and the partition 86.

According to this configuration, the drug solution L is introduced into the inner chamber 80 or the atomization structure of the spray cap 14, and then sprayed from the spray port 64. That is, the drug solution path in the spray cap 14 is only the inner chamber 80 and the spray port 64. As described above, in the above embodiment, the drug solution path in the spray cap 14 can be shortened. Therefore, the amount of the drug solution L in the drug solution path is small. Therefore, the amount of the drug solution L remaining in the drug solution path (inside the spray cap 14) after spraying the drug solution L decreases.

The spray cap 14 includes a core 66 provided with an atomization structure, the core 66 being accommodated in the inner chamber 80.

An example of such a core 66 is a porous body such as a sponge. Another example of the core 66 is a member in which a spiral groove is formed. Still another example of the core 66 is a member in which an orifice is formed. The drug solution L changes from liquid to mist in the process of flowing along the core 66. According to this configuration, it is easy to change the drug solution L into a mist form.

The core 66 is positioned and fixed to the spray cap 14 in the inner chamber 80.

In this case, it is easy to change the drug solution L having a predetermined viscosity into a mist form.

In another embodiment, the atomization structure is formed in the spray port 64a for spraying the atomized drug solution L in the spray cap 14.

In this configuration, it is not particularly necessary to accommodate the core 66 in the inner chamber 80. Therefore, it is easy to manufacture the spray cap 14.

The partition 86 is made of an elastic body.

The elastic body is relatively flexible. Therefore, in this case, the cap-side end portion 52 of the needle 18 easily penetrates the partition 86 and reaches the inner chamber 80. That is, according to this configuration, it is easy to cause the cap-side end portion 52 of the needle 18 to reach the inner chamber 80.

In another aspect, the partition 86a has an insertion portion 94. In the insertion portion 94, the needle 18 is inserted and removed as the spray cap 14 is attached to and detached from the barrel 16.

In this case, because the needle 18 passes through the insertion portion 94, the needle 18 does not penetrate the partition 86a. Therefore, there is no concern that the lumen 54 of the needle 18 is blocked by the fragment of the partition 86a.

The insertion portion 94 opens as the needle 18 is inserted into the inner chamber 80. On the other hand, the insertion portion 94 is closed as the needle 18 is detached from the inner chamber 80.

In this case, the insertion portion 94 opened as the needle 18 is inserted into the inner chamber 80 tries to return to the position before the needle 18 is inserted. Therefore, the inner wall of the insertion portion 94 comes into close contact with the peripheral wall of the needle 18 passed through the insertion portion 94. Therefore, leakage of the drug solution L flowing into the inner chamber 80 to the needle accommodating part 88 is avoided.

The partition 86 is a disk-shaped body.

In this case, the spray cap 14 is lightweight. In addition, in a case where the partition 86 is a disk-shaped body, it is relatively easy for the needle 18 to penetrate the partition 86.

In the spray device 100, the partition 106 is a filling portion for filling a space between the inner chamber 80 and the insertion port 84.

In this case, the partition 106 reliably blocks the drug solution L. Therefore, backflow of the drug solution L from the inner chamber 80 to the insertion port 84 is avoided. Therefore, leakage of the drug solution L from the insertion port 84 is avoided.

In the spray device 100, the partition 106, which is a filling portion, has a tapered hole 110 whose diameter expands in a tapered shape from the inner chamber 80 toward the insertion port 84.

The tapered hole 110 guides the cap-side end portion 52 of the needle 18 to a predetermined position of the partition 106. For example, an insertion hole 108 is formed at a predetermined position. In this case, it is easy to align the position of the needle 18 with the position of the insertion hole 108. Therefore, it is also easy to pass the needle 18 through the insertion hole 108.

In the spray device 122, the spray cap 120 is a variable capacity container which is changeable in capacity of the inner chamber 80.

By making the capacity of the inner chamber 80 variable, the drug solution L can be easily sprayed regardless of whether the drug solution L has a high viscosity or a low viscosity.

In the spray device 122, the spray cap 120 has a first segment 124 provided with an atomization structure and a second segment 126 provided with a partition 86. The spray cap 120 is configured by relatively movably connecting the first segment 124 to the second segment 126. The capacity of the inner chamber 80 is changed by the movement of the first segment 124 relative to the second segment 126.

According to this configuration, it is easy to change the capacity of the inner chamber 80 to a desired capacity.

Note that the present invention is not limited to the above disclosure, and various configurations can be adopted without departing from the gist of the present invention.

1. A spray device comprising:
a needled syringe comprising a barrel in which a drug solution storage chamber is formed, and a needle fixed to a distal end of the barrel; and
a spray cap attachable to the distal end of the barrel, wherein:
in a state in which the spray cap is attached to the distal end of the barrel, the spray cap accommodates the needle therein, and
the spray cap comprises an atomization structure configured to change a drug solution supplied from the drug solution storage chamber through the needle into a mist form, and to spray the drug solution changed into the mist form.

2. The spray device according to claim 1, wherein
the spray cap comprises:
an insertion port into which the distal end of the barrel is inserted,
an inner chamber formed on a distal end side of the insertion port and into which the drug solution supplied from the drug solution storage chamber flows, and
a partition that is interposed between the insertion port and the inner chamber, and
the distal end of the needle passes through the partition and reaches the inner chamber as the spray cap is attached to the distal end of the barrel, and the distal end of the needle is disposed between a proximal end of the atomization structure and the partition in an axial direction of the spray cap.

3. The spray device according to claim 2, wherein the partition is made of an elastic body.

4. The spray device according to claim 3, wherein the partition comprises an insertion portion into which the needle is inserted and removed as the spray cap is attached to and detached from the barrel.

5. The spray device according to claim 4, wherein the insertion portion is configured to open as the needle is inserted into the inner chamber and to close as the needle is detached from the inner chamber.

6. The spray device according to claim 2, wherein the partition is a disk-shaped body.

7. The spray device according to claim 2, wherein the partition fills a majority of a space between the inner chamber and the insertion port.

8. The spray device according to claim 2, wherein the partition has a tapered hole having a diameter that expands in a tapered shape from the inner chamber toward the insertion port.

9. The spray device according to claim 1, wherein:
the spray cap comprises:
an insertion port into which the distal end of the barrel is inserted, and
an inner chamber formed on a distal end side of the insertion port and into which the drug solution supplied from the drug solution storage chamber flows, and
the spray cap is a variable capacity container capable of changing a capacity of the inner chamber.

10. The spray device according to claim 9, wherein
the variable capacity container comprises:
a partition that is interposed between the insertion port and the inner chamber,
a first segment containing the atomization structure, and
a second segment containing the partition,
the first segment is connected to the second segment in a relatively movable manner, and
the first segment is movable relative to the second segment to change a capacity of the inner chamber.

11. The spray device according to claim 1, wherein:
the spray cap comprises:
an insertion port into which the distal end of the barrel is inserted,
an inner chamber formed on a distal end side of the insertion port and into which the drug solution supplied from the drug solution storage chamber flows, and
a core comprising the atomization structure, and the core is contained in the inner chamber.

12. The spray device according to claim 11, wherein the core is fixed to the spray cap within the inner chamber.

13. The spray device according to claim 1, wherein the atomization structure comprises a spray port for spraying the atomized drug solution in the spray cap.

14. A spray device comprising:
a needled syringe comprising a barrel in which a drug solution storage chamber is formed, and a needle fixed to a distal end of the barrel; and
a spray cap attachable to the distal end of the barrel, wherein:
in a state in which the spray cap is attached to the distal end of the barrel, the spray cap accommodates the needle therein, and
the spray cap comprises a core, wherein the core comprises a porous body, a spiral groove, or an orifice, and wherein the core is configured to change a drug solution supplied from the drug solution storage chamber through the needle into a mist form, and to spray the drug solution changed into the mist form.

15. A spray device comprising:
a needled syringe comprising a barrel in which a drug solution storage chamber is formed, and a needle fixed to a distal end of the barrel; and
a spray cap attachable to the distal end of the barrel, wherein:
in a state in which the spray cap is attached to the distal end of the barrel, the spray cap accommodates the needle therein, and
the spray cap comprises a spray port configured to change a drug solution supplied from the drug solution storage chamber through the needle into a mist form, and to spray the drug solution changed into the mist form.
